(19) United States
(12) Patent Application Publication
(10) Pub. No.: US 2014/0038276 A1
(43) Pub. Date: Feb. 6, 2014

(54) REACTION VESSEL

(71) Applicants: Joseph G. Cremonese, Greensburg, PA (US); Joseph E. Qualitz, Stow, MA (US); Brookman P. March, Harwick, PA (US); Michael Tolosa, Columbia, MD (US)

(72) Inventors: Joseph G. Cremonese, Greensburg, PA (US); Joseph E. Qualitz, Stow, MA (US); Brookman P. March, Harwick, PA (US); Michael Tolosa, Columbia, MD (US)

(21) Appl. No.: 13/837,290

(22) Filed: Mar. 15, 2013

Related U.S. Application Data
(60) Provisional application No. 61/678,312, filed on Aug. 1, 2012.

(51) Int. Cl. C12M 1/00

(52) U.S. Cl. CPC C12M 23/28
USPC 435/288.7; 435/289.1; 435/297.1

(57) ABSTRACT

We provide a miniature, disposable reactor vessel for bioprocessing. Embodiments include a sealed vessel surrounding a filter through which spent media may be preferentially removed relative to culture cells. Preferred embodiments include an impeller shaft that is contained within the vessel and passes through the filter assembly. The impeller shaft may be engaged magnetically with a drive shaft. Combinations of these reactor vessels and methods of their use are also described.

### CROSS-REFERENCE TO RELATED APPLICATIONS

This application claims priority to U.S. Provisional Patent Application No. 61/678,312, filed on Aug. 1, 2012, and incorporated by reference herein.

### BACKGROUND OF THE INVENTION

1. Field of the Invention

Embodiments of the invention relate to small bioprocess reaction vessels that offer similar functionality to that of large stirred-tank production-sized vessels used for the production of biopharmaceuticals, including therapeutic proteins, polysaccharides, vaccines and diagnostics; specialty products, including antibiotics, low molecular weight pharmaceutical chemicals; value added food and agriculture products; and fuels, chemicals and fibers from renewable resources.

2. Description of the Related Art

Bioprocessing methods have been around since the beginning of recorded history. As early as 2000 B.C. the Egyptians documented the brewing of beer. The use of yeast cells to produce CO₂ before and during the bread baking process has been practiced through history. Fermentation techniques have been used by our ancestors to produce wine, balsamic vinegar, soy sauce, and other food additions. In 1857, Louis Pasteur showed that lactic acid fermentation was caused by living organisms. In 1860, Pasteur demonstrated that bacteria caused the souring of milk. In 1877, Pasteur published “Etudes sur la Biere” and correctly showed that specific types of microorganisms cause specific types of fermentations and result in specific end-products.

German chemist Eduard Buechner ground up yeast, extracted a juice, and found that the juice would ferment a sugar solution, forming CO₂ and alcohol, much like living yeast would. From that time, the word “enzyme” came to be applied to all ferments. That initiated the understanding that fermentation is caused by enzymes produced by microorganisms. In 1912, Alexis Carrel, a French physician and surgeon, experimented with mammalian cell cultures starting with embryo chick heart cells. He found that by nourishing the cells by changing the nutrient media regularly that the cells would continue to grow in a unique laboratory culture flask invented by Carrel. It is thought now that Carrel's experiment became contaminated with ‘immortal’ cells in the process. The point is that Carrel kept the cells alive and growing for 20 years.

Partly as a result of Carrel's experiments, cell culturing as a laboratory technique evolved. The Carrel Flask became a tool that facilitated the growth of mammalian cells by simplifying the process of removing metabolic wastes from the mammalian cultures and replacing the spent nutrient media with fresh nutrient media. As cells grew and became confluent in the Carrel Flask, it became apparent that the cells could be harvested and the cells remaining behind would once again become confluent as long as the nutrient media was regularly replaced. Carrel's technique advanced so that new cell lines were established and cell culturing as a laboratory science was established. Removing metabolic waste and ‘feeding’ cell cultures with fresh nutrient media was time consuming and labor intensive. It required that the Carrel Flask be taken to a containment hood and the changing of the nutrient media be done under aseptic conditions so as not to contaminate the growing cells in the process.

The Carrel Flask facilitated the harvesting, study and utilization of mammalian cells. That led, after several years of experimenting, directly to the use of metabolic byproducts of the cells. It also led to large scale fermentation methods for the production of certain metabolic products, i.e. hormones, by utilizing bacterial cells as microscopic “factories.” Since bacterial cells such as E. coli proliferate so much more rapidly than mammalian cells, large scale fermentation methods developed faster and became more common in bioprocess development. And, the process has been substantially the same for many years. Large fermentation tanks became known as bioreactors.

Knowing and understanding the significance of pH and dissolved oxygen content of the growing cultures would become pivotal for controlling the growth and health of the cultures. Temperature control was very important. Stirring the cultures helped with the aeration and suspension of the cultures. Cell growth characteristics required that air and oxygen be added to the cultures to achieve a predetermined cell growth environment for healthy living cells. Fresh media addition is a requirement for longer growth time cultures. The addition of CO₂ gas and liquid base material are used to adjust pH. Control devices were developed to add the required gas, nutrient media or base to the cultures.

Advances in microbiology and fermentation technology continued steadily and, by 1978, it was discovered that microorganisms could be mutated with physical and chemical treatments to develop higher producing, faster growing strains, tolerant of less oxygen, and able to use more concentrated medium. Strain selection and hybridization have come to be important considerations in the development and optimization of bioprocessing methods.

Since the completion of the Human Genome Project in the year 2000, it has been estimated that there are about 3 million proteins in the make-up of human and animal cells and fewer than 10% have been characterized to date. From the genome mapping alone, emerging protein therapies will add to the huge backlogs of products arising from the unfulfilled demands of the already 20 year-old recombinant-DNA technologies and hybridoma technologies. Emerging biopharmaceuticals are creating huge and ever-growing needs for faster and faster bioprocess development and optimization. Currently most protein expression is by E. coli, bacculovirus or similar systems.

Mammalian cells, used for bioprocessing, have the advantage of being able to produce complex, bioactive molecules. However, mammalian cells grow and express proteins at approximately 5% of the rate of E. coli cells. Mammalian cells also require expensive growth media. The use of mammalian cells requires higher capital and labor costs. There is an unfulfilled demand for faster and less costly high throughput mammalian culture methods for bioprocess development and bioprocess optimization.

Bioprocessing has become so completely embedded in our lives today so that, without bioprocessing, millions and millions would starve or we'd drown in our own waste. Bacteria, enzymes, proteins and various other biological materials are processed in manufacturing facilities. Bioprocessing is responsible for the production of many healthcare and well-being products including insulin, growth hormones, replacement hormones, alpha-interferon, monoclonal antibodies, hepatitis vaccine, erythropoietin, vitamins, chemotherapeutics, hyaluronic acid, laboratory diagnostics materials i.e. tissue plasminogen activator, cardiac enzyme tests, liver function tests, cancer screening tests, thyroid screening tests and the lists go on and on. Protein characterization, genetic mapping, DNA analysis, and cell and tissue typing, are but a few of the diagnostic developments that have arrived in the last half of the twentieth century.

Biological waste treatment is now the norm for cleansing water, removal of gas and clearing of odors. Oils and fats are processed using bioprocess methods. Biodegradable plastics are possible today because of bioprocessing. Agricultural plants can be engineered to synthesize therapeutic proteins. Bioengineering has produced crops that can fight off disease and crops with higher protein content. Perhaps the fastest growing technology today has to do with the discovery of new therapeutics and medicines as a result of bioprocess technology. New treatments for anemia and leukemia are produced with biopharmaceutical processing. Customized cancer treatments are starting to emerge as a result of biotechnology.

Production scale growth of mammalian cells or microbial cells is commonly performed by pharmaceutical companies in very large, primarily stainless steel, stirred tank reactors for the purpose of producing biopharmaceuticals. The use of such very large production vehicles is not a practical option for process development or process optimization. For many years, laboratory bench-scale process development and process optimization has been carried out in scalable bioreactors that have customarily ranged in size from 1 liter volumes to 5 liter volumes. Laboratory bench-scale bioprocessing, while an effective alternative for process development and optimization, is still somewhat labor intensive and time consuming if you consider the sterility requirements as well as the reactor set-up time when the need is for a multiplicity of parallel experiments.

Since the year 2000, there has been explosive growth in the pursuit of new biopharmaceuticals. High-throughput bioprocessing is a promising technique for the development and optimization of mammalian and microbial cultures. There is a demand to significantly reduce time and material and labor costs by using mini-sized reaction vessels without losing the ability of scale-up or process applicability in predicting the much larger production scale reactions. There is also the desire to greatly reduce hands-on time for experiment set-up and the time for replication of a multiplicity of parallel reactions necessary to prove process engineering scale-up calculations.

It is becoming, at the least, increasingly more difficult and, at the worst, impossible to keep up with the demands for process development with the development and optimization systems in place today. Process development, currently and for the most part, is done in volumes of 1 to 5 liters because the scale-up characteristics are well documented. It has been done that way for thirty years or more. The increasing costs of continuing with current methods for process development and process optimization are starting to outstrip our ability to fund new process development. It has become imperative that we reduce the size of the reaction vessels to reduce the costs of materials required for the quantity of multiple and parallel process experiments necessary for process development. It has become imperative to reduce the operating hardware clean-up time, sterilization requirements, and turn-around time. And, increasing labor costs have outpaced the costs of materials disposability.

### BRIEF SUMMARY OF THE INVENTION

We provide a disposable miniature reaction vessel that can eliminate time-consuming set-up and repetitive sterilization requirements for bioprocess experimentation while retaining reaction scale-up predictability. Embodiments may have the ability to mimic the cell growth characteristics of large-scale stirred tank reactors. Further embodiments may significantly reduce the volume of nutrient media required to observe cell growth rates in parallel reactions. This leads to time and labor savings, particularly in light of the disposability of embodiments of the vessel. This eliminates the need for disassembly, cleaning, reassembly, sterilization and finally set-up of the sterilized vessel for re-use. Embodiments may provide a very small volume bioreactor vessel that cooperates with a process control module that responds quickly, in real-time, to adjust to the changing metabolic demands of the cell growth process, independently in each individual reaction vessel, and in the immediate and automatic corrections in the process parameters, independently in each individual reaction vessel

We report reaction vessel comprising a reactor containment shell. The reactor containment shell comprises a cylindrical outer wall having a top opening and a bottom opening and defining a shell interior; a vessel top covering the top opening; a drive shaft receptable defined by a second cylindrical wall on the vessel top, opposite the shell interior; an impeller shaft receptacle defined by a third cylindrical wall on the vessel top, extending partially into the shell interior; a securing receptable defined by a fourth cylindrical wall on the vessel top, said fourth cylindrical wall extending partiall into the shell interior to a distance greater than the impeller shaft receptacle extends, said securing receptacle concentric with and having a greater diameter than the impeller shaft receptacle; and a vessel bottom covering the bottom opening.

In further embodiments the vessel top further comprises a plurality of access ports permitting access to the shell interior from outside the reaction vessel. At least one of the access ports may be connected to a closed cannula extending into the shell interior. One or more of the access ports may lead to a least one member of the group consisting of an oxygen sparger, an air sparger, a nitrogen inlet, a carbon dioxide inlet, a cell harvest tube, a liquid addition inlet, a vent, an overlay gas inlet, a drawoff tube, and a thermowell.

In most embodiments the reaction vessel further includes a filter assembly. The filter assembly may include an inner cylinder concentric with an outer cylinder, both resting atop a base plate, covered by a flange, and defining between them a waste receptacle, said base plate and said flange each including a hole aligned with the hole in the other such that an impeller shaft may pass through them and the inner cylinder simultaneously; said base plate including a collar in which the inner cylinder is secured and outside of which the outer cylinder is secured; wherein the base plate has an edge extending beyond the outer cylinder, and wherein the outer cylinder includes at least one opening in its wall; and a filter disposed about the outer concentric cylinder, said filter resting on the base plate; wherein said flange is disposed within said securing receptacle and abuts said impeller shaft receptacle.

The filter may allow passage of media having a diameter less than between 0.1 to 0.8 microns. The outer cylinder may include four openings at 90 degree intervals about the circumference of the outer cylinder, wherein said openings are longitudinal openings.

The reaction vessel may also include an impeller shaft, said impeller shaft having a drive end and an impeller end, said drive end comprising at least two magnets capable of engaging a drive shaft when a drive shaft is disposed in said drive shaft receptacle.

An impeller shaft may be included. An impeller shaft typically has a drive end and an impeller end, said drive end comprising at least two magnets capable of engaging a drive shaft when a drive shaft is disposed in said drive shaft receptacle.

Reaction vessels of preferred embodiments of the invention have a volume 50 mL and 150 mL. Reaction vessels may also include at least one sensor in the vessel bottom and in communication with the void. The sensor may be a fluorescent sensor. It may help detect, for example, pH, dissolved oxygen level, carbon dioxide level, and temperature.

Reaction vessels may further include at least one tube for applying partial vacuum to the waste receptacle. Reactor assemblies are also provided. They may include a control module and at least one reaction vessel.

Other details, objects, and advantages of the invention will become apparent as the following description of certain present preferred embodiments thereof proceeds.

### DETAILED DESCRIPTION OF THE FIGURES

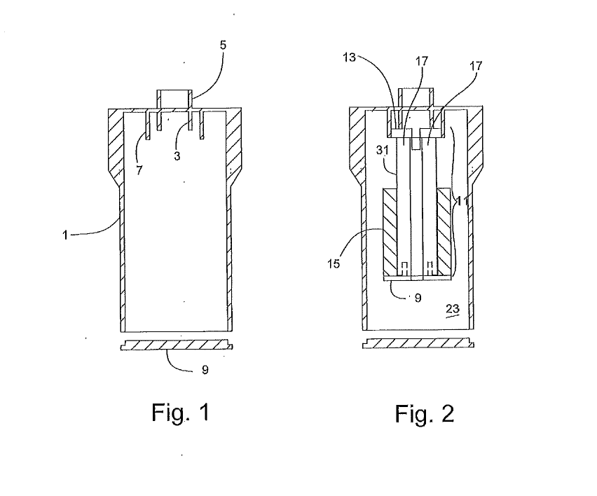

FIG. 1 shows a longitudinal cross-section of a reaction vessel of an embodiment of the invention.

FIG. 2 illustrates a cross section of a reaction vessel including a filter holder/waste media reservoir (also referred to herein as a “filter assembly”) of an embodiment of the invention.

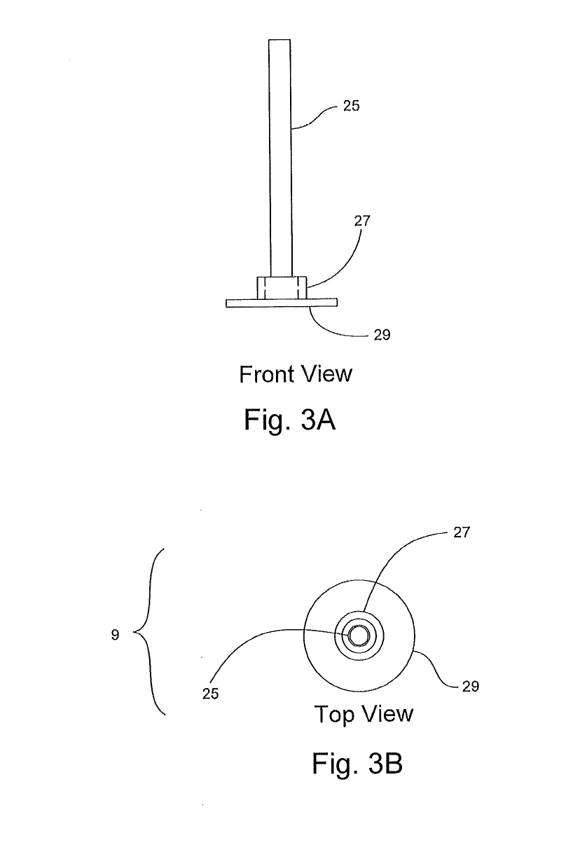

FIG. 3A shows a front view of, and FIG. 3B shows a top cross-sectional view of, the filter assembly closure illustrating the waste reservoir isolation tube sealed into the base.

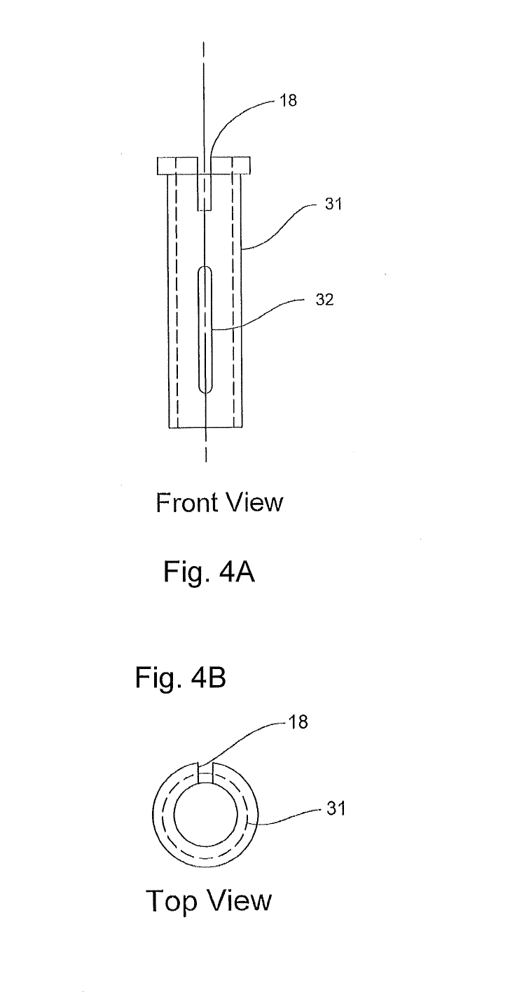

FIG. 4A illustrates a front view of, and FIG. 4B a top view of, the filter assembly's outer body tube.

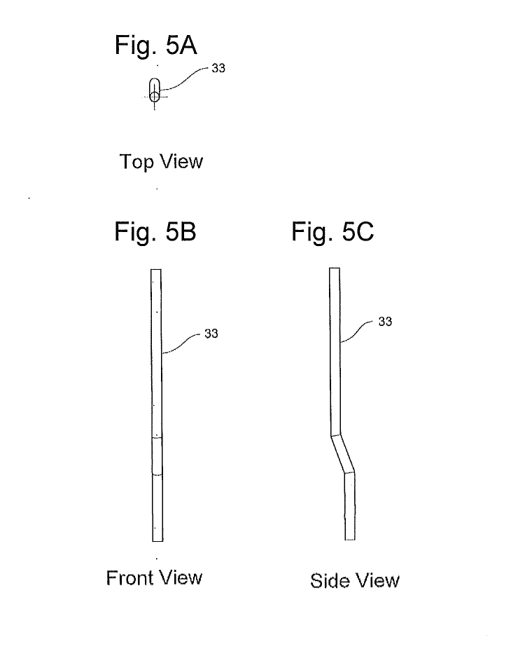

FIG. 5A, FIG. 5B, and FIG. 5C show, respectively, a top view, front view, and side view of the waste draw-off tube positioned within a isolated waste reservoir.

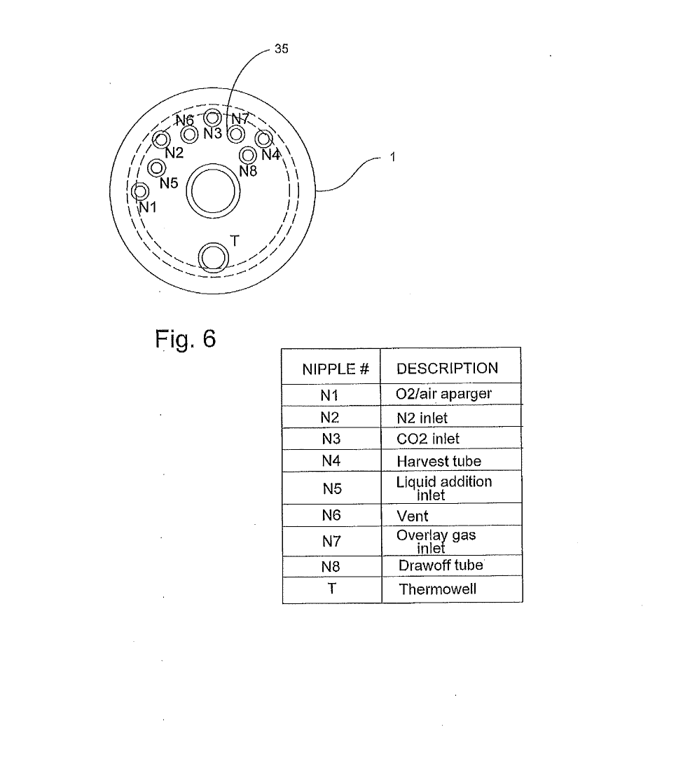

FIG. 6 illustrates a top view of a single-use reaction vessel of an embodiment of the invention with the positioning of access ports noted.

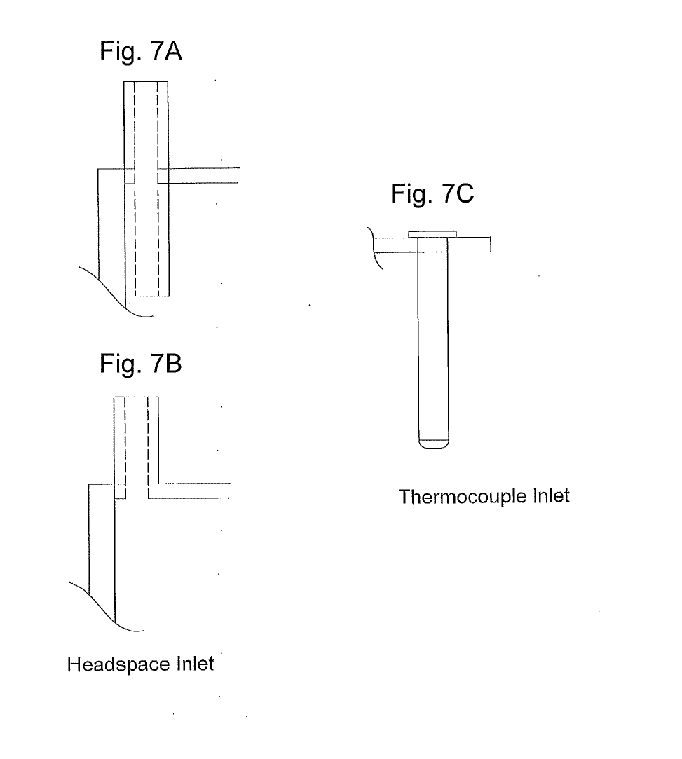

FIG. 7A, FIG. 7B, and FIG. 7C illustrate longitudinal views of inlet access ports.

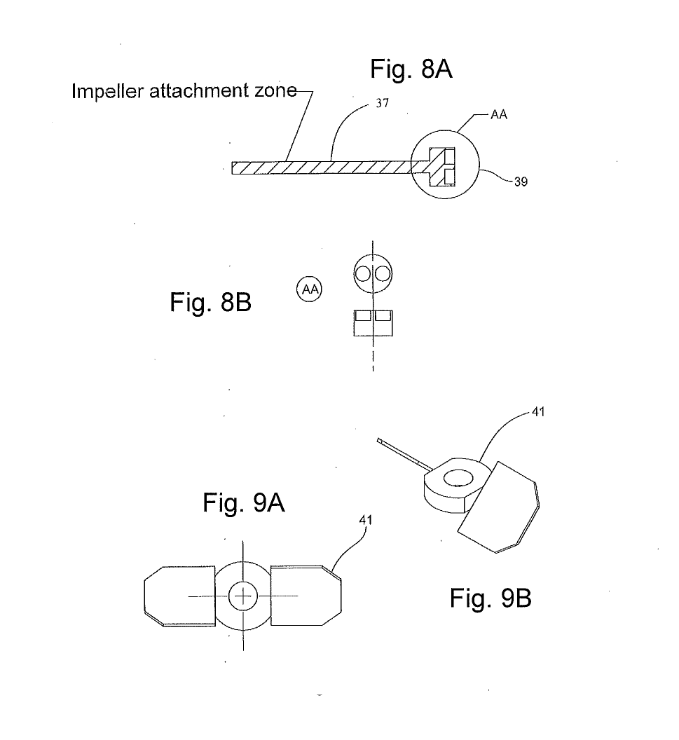

FIG. 8A illustrates an impeller shaft used in a embodiment of the invention. FIG. 8B shows a side-view detail of a portion of the impeller labelled “AA.”

FIG. 9A and FIG. 9B show two cell-lifting impellers that are press-fitted on an impeller shaft.

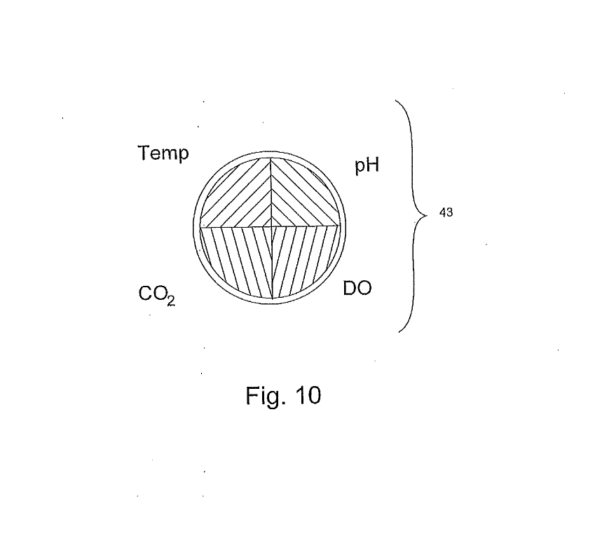

FIG. 10 shows the top surface of the bottom closure (see also FIG. 1 and FIG. 2) of an embodiment of the invention.

### DETAILED DESCRIPTION OF THE INVENTION

Embodiments of the invention provide a reaction vessel (vessel) that will mimic the reactions occurring in large, stirred tank, production-sized vessels. Preferred embodiments of the invention are small volume, and are disposable. Typical embodiments of the invention are operated in conjunction with a control module. The control module modulates the addition of gas or liquid media and regulates the speed of culture stirring impellers as needed to achieve optimum cell growth conditions. The control module also controls reaction vessel temperature.

Typically the control module is used to provide desired cell growth conditions. These may operate the reaction vessel, for example by directing gas valves to open and close, and liquid pumps to start and stop thereby adjusting the culture contents, within the vessel, to optimum conditions. The control module may include a magnetic stirring drive mechanism that cooperates with magnets on the impeller of the reaction vessel. This, with proper placement of a magnetic shaft driving disc, and placement of the impellers within the disposable vessel, allows the impeller shaft to be rotated without penetrating the reaction vessel.

The reaction vessel is designed to reside within a well in a thermally controlled aluminum block for achieving optimal temperature conditions of the residing culture, independently, within each reaction vessel. In a preferred embodiment when each reaction vessel is disposed within each thermal well, the reaction vessel is also in direct alignment with an external optical system that provides frequency modulated light emission, at timed intervals, to excite sterile disposable sensor pads placed inside each aseptically protected reaction vessel.

As discussed more fully below, the reaction vessel may include a sensor pad or pads. These sensor pads, influenced by the internal conditions of each individual reactor vessel, respond to the exciter beam by emitting a fluorescent wavelength response, the frequency of which is filtered with an optical filter and the intensity detected by an optical detector, which then signals the condition of the pH and dissolved oxygen, within the culture, to the control module for comparison to the optimum data as programmed by the operator. The comparison of actual conditions to desired conditions drives the response of each individual gas valve or liquid pump to adjust the gas or liquid additions to each vessel independently of other reactor vessels that may be operated in parallel. In one embodiment of the invention a single control module may include software that can control 12 reaction vessels simultaneously and independently.

The single-use reaction vessel will be designed to accommodate protocols for mammalian cell cultures, insect cell cultures, and fermentation methods using E. coli, yeast cells or other cells for fermentation methods. The reaction vessels may be fitted out with components as necessary for static cultures, perfusion methods, or “fed-batch” methods. Although the reaction vessel has been described repeatedly as “disposable” or “single use,” it should be understood that such a description describes only some, not all, of the embodiments of the invention.

In fed-batch methods, nutrient media is added to replace an equal volume of spent media. The primary objective of “fed-batch” processing in this single-use reaction vessel is to continue to increase cell density without changing the working volume of the culture medium and cells within the reaction vessel. This requires the removal of and replacement of spent media without disturbing the cell density. To accomplish this with a small (100 mL) working volume vessel report herein, one may pull spent media, but not cells, through a filter to a central reservoir and then evacuate the reservoir volume to an external waste receptacle. The rate of flow of fresh nutrient media, entering the vessel, meets but does not exceed the rate of flow of spent media being evacuated, thereby maintaining the integrity of the working volume.

To maintain balance in the working volume, we provide a filter holder/reservoir combination that allows filtration of the culture medium and removal of spent media with a design that provides a passage tunnel through the central part of the filter holder such that the impeller shaft passes through the tunnel without the need for liquid seals or shaft bearings.

Embodiments of the invention may be better understood through reference to the figures. FIG. 1 illustrates a cross-section of a cylindrical reactor containment shell 1 including inner receptacle 3 and securing receptacle 5. The cylindrical reactor containment shell may be a right circular cylinder or may have another cross-section at the selection of a user. The receptacles are at the top of the vessel and juxtaposed to allow coupling of an external drive shaft (not shown in FIG. 1) in securing receptacle 5 and an internal impeller shaft (not shown in FIG. 1) seated in inner receptable 3 without the need for a penetrable bearing or opening in the top of the vessel. FIG. 1 also illustrates, larger than and concentric to the internal impeller shaft receptacle, a concentric receptacle 7 for receiving a filter holder/waste media reservoir. The bottom vessel closure 9 is also illustrated. This may be retained, for example, by adhesive or heat welding. This allows the interior of the vessel to be isolated from the environment.

Production of the reactor containment shell may be accomplished by any convenient method. Suitable methods include, for example, injection molding, vacuum forming, and other common means of mass fabrication.

Although various embodiments are described herein in the context of the further addition of a filter holder/waste reservoir to the containment shell of the reactor vessel, those skilled in the art will recognize that the reactor vessel may be useful without the filter holder/waste reservoir (interchangably referred to herein as the “filter assembly”). This may particularly be the case when the reservoir is used for static cultures.

FIG. 2 shows a cross-section of a cylindrical filter assembly 11 used in embodiments of the invention. The assembly acts as a combination filter holder and waste reservoir. As shown in FIG. 2, the filter assembly 11 is pressed into the concentric receptacle 7, with the upper flange 13 of the filter assembly abutting the inferior portion of the central impeller shaft receptacle and the assembly held in place with an interference fit. FIG. 2 also illustrates a longitudinal section of the cylindrical filter 15 as fitted over the filter assembly. Waste Reservoir 17 is included in the assembly. The bottom filter support/reservoir closure 19 is also illustrated. With the bottom closure in place, the waste reservoir is isolated from the impeller shaft tunnel 19 and the medium within the reaction vessel. The cell culture and growth media reside in void 23. Four longitudinal channels (not shown in FIG. 2), placed at 90 degrees apart, penetrate the outer concentric wall of the filter holder such that the only access to the inner waste receptacle is through the filter.

FIG. 3(A) shows a front detail of the filter assembly closure 19. FIG. 3(B) shows a top cross-sectional view of the filter assembly closure illustrating the waste reservoir isolation tube sealed into the base. The isolation tube 25, sealed into a collar 27 of the base plate 29, allows an impeller shaft to pass through unrestricted and without the need for a penetrable bearing.

FIG. 4(A) illustrates a longitudinal detail of the filter assembly outer body tube 31. FIG. 4(B) illustrates a top view of the filter assembly outer body tube 31. FIG. 4(A) also shows a longitudinal channel 32 that penetrates the outer concentric wall of the filter holder and allows access to the waste reservoirs 17. In preferred embodiment four such channels are present and are spaced equidistantly about the circumference of the outer body tube. FIG. 4(B) also shows the location of an access slot 18 for the entry and positioning of a waste draw-off tube that may reside within the isolated waste reservoir.

FIGS. 5(A), 5(B), and 5(C) show top, front, and side views, respectively, of a waste draw-off tube 33 that will be positioned within the isolated waste reservoir and connected to the evacuation port that is molded into the top of the single-use reaction vessel. This tube allows removal of spent media from the waste receptacle.

FIG. 6 shows a top view of a single-use reaction vessel of an embodiment of the invention, including a plurality of access ports 35. These access ports may have multiple functions, including as access points for addition of gasses to the system, addition of liquids, venting, testing of temperature, sparging, harvesting of cells, or heating. In some embodiments the access ports are attached to nipples that permit them to be attached to hoses. In some embodiments the access ports are connected to tubes that extend for a distance into the vessel, allowing materials to be placed in or removed from various levels in the vessel. In some cases the inlets extend only to the headspace of the container.

Some embodiments of the invention include, attached to a port, an “L” shaped stainless steel cannula, connected to a port molded into the underside of the topmost surface of the single-use vessel such that it can deliver air and/or oxygen sparge gas through micro-pore sized holes fabricated into the internally projecting arm of the cannula. The gas bubbles emerging from the arm are of a uniform size and are directed vertically from bottom to top passing through the liquid media in the aseptic chamber when vessel is in use. Useful sparge gas cannula are reported, for example, in Kondragunta, et al., “Bioprocess Convergence Using Sentinel Genes for Process Parameter Tuning,” Biotech. Progress, (2012), 28(5), 1138-1151.

The positioning of access ports in an embodiment having six ports is noted in FIG. 6, and the ports are labelled as shown in the chart accompanying the figure. The four outermost access ports enter the top of the vessel and run to within several millimeters of the bottom of the vessel. One of the outermost ports in the embodiment shown is for liquid media entry and cell inoculation entry into the single-use vessel. Details of various access ports are shown in FIGS. 7(A), 7(B), and 7(C).

FIGS. 8(A) and 8(B) illustrate an impeller shaft 37. The impeller shaft is enclosed by the vessel 1 and seated in inner receptacle 3. It travels through impeller shaft tunnel 19 into the mixture of media and cells in void 23. To avoid communication of the impeller shaft with the environment outside the vessel, at its top the impeller shaft includes magnets 39 that allow it to couple with the a drive shaft outside the vessel. The drive shaft also includes two magnets, located outside the vessel. Typically these magnets are rare earth magnets. In one embodiment they are neodymium magnets.

FIGS. 9(A) and 9(B) show two cell-lifting impellers 41 that may be press-fitted or crimped onto the impeller shaft. In a preferred embodiment an uppermost impeller is located several millimeters inferior to the lowermost part of the filter assembly, and the lowermost impeller is located about 5 millimeters superior to the distal end of the stirring shaft.

FIG. 10 shows the top surface of the bottom vessel closure 9 of an embodiment of the invention. When the bottom vessel closure is attached to the vessel, a sensor 43 is located inside the vessel. The top surface of the sensor, as shown in FIG. 10, shows an embodiment in which four separate sensor patches are placed on a sensor. Sensor patches appear as pie-wedge sections, though concentric circles or other shapes are possible. They are in contact with the internal environment of the vessel and visible from outside the vessel. Vessels may contain 1, 2, 3, 4, or more sensor patches. In a preferred embodiment the four sensor patches measure pH, Dissolved Oxygen, CO₂, and Temperature.

These sensors offer a number of advantages. For example, they may be continuously responsive, and they may provide information in real-time using fluorescence. Preparation and use of suitable sensors are described, for example, in Ge, et al., “Validation of an Optical Sensor-Based High-Throughput Bioreactor System for Mammalian Cell Culture,” J. Biotech. 122 (2006) 293-306, and Hanson, et al., “Comparisons of Optical pH and Dissolved Oxygen Sensors with Traditional electrochemical Probes During Mammalian Cell Culture,” Biotech & Bioeng. 97:4 (2007) 833-841. Both of those documents are incorporated by reference herein. When in use, the sensors may be positioned over a fluorometer or mini-fluorometer so that when light is emitted from the fluorometer it excites the non-invasive sensors, causing a response that is influenced by the pH and amount of dissolved oxygen in the aseptic interior of the vessel.

The results of the sensors may be logged. In preferred embodiments, the logged results are used to provide a feedback loop that will allow conditions in the reaction vessel to be modified based on the detected conditions.

Operation of a reactor vessel according to embodiments of the invention is straightforward. The bottom of the vessel may be sealed either before or after the vessel is filled with a mixture of cells and growth media. As the cells grow, media is driven through the filter and into the waste receptacle for removal from the vessel. Typically the cells are larger than the maximim size of particle that is allowed to pass the filter, preventing their removal except through one or more of the ports designed for that purpose.

As spent media is removed through the filter, additional media is added through one or more ports. Typically the addition occurs through a port that includes a cannula that allows the fresh media to be added to the bottom of the vessel, preventing its immediate removal through the filter and allowing it to be raised through the vessel by the impeller.

Suitable filters may depend on the type of cell culture being prepared. Normally a filter has pore sizes between 0.1 microns to 0.8 microns.

We provide a number of ways to evacuate the waste reservoir. For example, it may be evacuated through a peristaltic pump, syringe pump, or vacuum pump with trap. Typically the waste reservoir is evacuated through a draw-off tube externally connected through an evacuation port in the head-plate of the vessel. In some embodiments a peristaltic pump is used to synchronize the rate of addition of fresh media with the rate of draw-off of spent media. This allows maintenance of a constant working volume within the vessel. This, in turn, allows the reaction to be maintained for significantly longer than is often possible with other methods and vessels. In some embodiments the cell growth may be maintained for between 14 to 20 days.

Typical reaction vessels of the invention have a volume of 100 mL. In some embodiments they have a volume of between 15-150 ml. In other embodiments they have a volume of between 100-500 ml.

Use of these small-volume reaction vessels provides substantial advantages over larger vessels. For example, we may provide a plurality of vessels running in parallel without the substantially increased footprint of a larger system. In one embodiment twelve 100 ml reactor vessels are run in parallel, allowing simultaneous collection of reaction data. this allows use of a total starting volume of only 1.2 L of culture medium, compared to the 6.0 L of culture medium that would be necessary if 500 mL reactors were used in any significant number in an attempt to obtain similar amounts of comparative data.

In some embodiments the removal of spent media is enhanced by creation of a partial vacuum in the waste reservoir. Pulling a partial vacuum in the waste reservoir increases passage of spent media from the vessel, through the filter, and into the reservoir. When a partial vacuum is expected to be applied it will typically be positioned so that it draws from as close to the top of the waste reservoir as possible, and a draw-off tube for removal of the spent media will be positioned so that it draws from the bottom of the waste reservoir.

While we have shown and described certain present preferred embodiments of our invention and have illustrated certain present preferred methods of using the same, it is to be distinctly understood that the invention is not limited thereto but may be otherwise variously embodied and practiced within the scope of the following claims.

Patents, patent applications, publications, scientific articles, books, web sites, and other documents and materials referenced or mentioned herein are indicative of the levels of skill of those skilled in the art to which the inventions pertain, as of the date each publication was written, and all are incorporated by reference as if fully rewritten herein. Inclusion of a document in this specification is not an admission that the document represents prior invention or is prior art for any purpose.

We claim:

1. A reaction vessel comprising:
a reactor containment shell comprising:
a cylindrical outer wall having a top opening and a bottom opening and defining a shell interior;
a vessel top covering the top opening;
a drive shaft receptable defined by a second cylindrical wall on the vessel top, opposite the shell interior;
an impeller shaft receptacle defined by a third cylindrical wall on the vessel top, extending partially into the shell interior;
a securing receptable defined by a fourth cylindrical wall on the vessel top, said fourth cylindrical wall extending partiall into the shell interior to a distance greater than the impeller shaft receptacle extends, said securing receptacle concentric with and having a greater diameter than the impeller shaft receptacle; and
a vessel bottom covering the bottom opening.

2. The reaction vessel of claim 1, said vessel top further comprising a plurality of access ports permitting access to the shell interior from outside the reaction vessel.

3. The reaction vessel of claim 2, wherein at least one of said access ports is connected to a closed cannula extending into the shell interior.

4. The reaction vessel of claim 2, wherein the access ports lead to a least one member of the group consisting of an oxygen sparger, an air sparger, a nitrogen inlet, a carbon dioxide inlet, a cell harvest tube, a liquid addition inlet, a vent, an overlay gas inlet, a drawoff tube, and a thermowell.

5. The reaction vessel of claim 1, further comprising a filter assembly, said filter assembly comprising:
an inner cylinder concentric with an outer cylinder, both resting atop a base plate, covered by a flange, and defining between them a waste receptacle, said base plate and said flange each including a hole aligned with the hole in the other such that an impeller shaft may pass through them and the inner cylinder simultaneously;
said base plate including a collar in which the inner cylinder is secured and outside of which the outer cylinder is secured;
wherein the base plate has an edge extending beyond the outer cylinder, and wherein the outer cylinder includes at least one opening in its wall; and
a filter disposed about the outer concentric cylinder, said filter resting on the base plate;
wherein said flange is disposed within said securing receptacle and abuts said impeller shaft receptacle.

6. The reaction vessel of claim 5, wherein the filter allows passage of media having a diameter less than between 0.1 to 0.8 microns.

7. The reaction vessel of claim 5, wherein the outer cylinder includes four openings at 90 degree intervals about the circumference of the outer cylinder, wherein said openings are longitudinal openings.

8. The reaction vessel of claim 1, further comprising an impeller shaft, said impeller shaft having a drive end and an impeller end, said drive end comprising at least two magnets capable of engaging a drive shaft when a drive shaft is disposed in said drive shaft receptacle.

9. The reaction vessel of claim 5, further comprising an impeller shaft, said impeller shaft having a drive end and an impeller end, said drive end comprising at least two magnets capable of engaging a drive shaft when a drive shaft is disposed in said drive shaft receptacle.

10. The reaction vessel of claim 1, said reaction vessel having a volume between 50 mL and 150 mL.

11. The reaction vessel of claim 1, further comprising at least one sensor in the vessel bottom and in communication with the void.

12. The reaction vessel of claim 11, wherein said at least one sensor is a fluorescent sensor.

13. The reaction vessel of claim 10, wherein said fluorescent sensor detects at least one member of the group consisting of pH, dissolved oxygen level, carbon dioxide level, and temperature.

14. The reaction vessel of claim 1, further comprising a cell culture in the void.

15. The reaction vessel of claim 5, further comprising at least one tube for applying partial vacuum to the waste receptacle.

16. A reactor assembly comprising:
a reactor control module; and
at least one reaction vessel of claim 1.
